(19) United States
(12) Patent Application Publication
(10) Pub. No.: US 2006/0127348 A1
(43) Pub. Date: Jun. 15, 2006

(54) Chemically and/or biologically reactive compounds

(75) Inventors: Henry Lomasney, Pagosa Springs, CO (US); Christina Lomasney, Sammamish, WA (US); John Grawe, West Palm Beach, FL (US)

Correspondence Address: HELLER EHRMAN WHITE & MCAULIFFE LLP, 1717 RHODE ISLAND AVE, NW, WASHINGTON, DC, 20036-3001, US

(73) Assignee: Isotron Corporation, Seattle, WA (US)

(21) Appl. No.: 10/513,174

(22) PCT Filed: May 2, 2003
(86) PCT No.: PCT/US03/13713

§ 371 Date: Nov. 14, 2005

Related U.S. Application Data
(60) Provisional application No. 60/376,804, filed on May 2, 2002.

(51) Int. Cl. A61K 31/785; A61K 31/765

(52) U.S. Cl. 424/78.3; 977/915

(57) ABSTRACT

This disclosure provides novel compositions comprising an inorganic and organic compound, which provides a means for the indirect attachment of a reactive species, such as an organic reactive molecule, within a binder polymer matrix. Such compositions provide a hydrophilic nanoscale domain that is uniformly dispersed within the polymer matrix. The nanoscale domain comprises inorganic particles having a nanoscale dimension. Such compositions can enhance the performance potential of the re-active species within the polymer material. The polymer composite that results from the introduction of such reactive species into a polymer matrix provides a self-decontaminating feature. The reactive species include those that are capable of associating with a halogen to form a complex that is active in decontamination of chemical or biological agents.

This application claims priority to U.S. Provisional Application No. 60/376,804 filed May 2, 2002, the entirety of which is hereby incorporated by reference.

### 1. Field of the Invention

This disclosure relates to decontaminating compositions and their use in decontaminating chemical or biological agents. Embodiments of the decontaminating compositions comprise an inorganic nanoscale domain, which comprises an inorganic nanoparticle and an organic reactive molecule grafted via a linker group onto the inorganic nanoparticle. The inorganic nanoscale domain may be attached to and uniformly dispersed within an polymer matrix and the composition can optionally be configured to be decontaminating upon contact, catalytically reactive or rechargeable. One such rechargeable species involves activation by contact with a halogen.

### 2. Description of the Related Art

Current disinfectants which are used for disinfecting purposes such as disinfecting water, potable water supplies, swimming pools, hot tubs, industrial water systems, cooling towers, spacecraft, waste water treatment plants, air conditioning systems, military field units, camping expeditions, and in other sanitizing applications, as well as of organic fluids, such as oils, paints, coatings, and preservatives, and in various medicinal applications, all have serious limitations. The most commonly used disinfectant, free halogens (chlorine, bromine, or iodine) are effective disinfectants, but free halogen is corrosive toward materials, toxic to marine life, reactive with organic contaminants to produce toxic trihalomethanes, irritating to the skin and eyes of humans, and relatively unstable in water, particularly in the presence of sunlight or heat. Ozone and chlorine dioxide are also effective disinfectants, but they are not persistent in water such that they have to be replenished frequently; they also may react with organic contaminants to produce products having unknown health risks. Combined halogen compounds such as the commercially employed hydantoins and cyanurates as well as the recently discovered oxazolidinones (Kaminski et al., U.S. Pat. Nos. 4,000,293 and 3,931,213) and imidazolidinones morley et al., U.S. Pat. Nos. 4,681,948; 4,767,542; 5,057,612; 5,126,057) are much more stable in water than are free halogen, ozone, and chlorine dioxide, but in general they require longer contact times to inactivate microorganisms than do the less stable compounds mentioned.

Further reactive species, such as free halogens, are effective disinfectants, but they are corrosive toward polymer materials. Shortly after World War II, the United States military devised and deployed a technology that addressed the corrosive behavior of the halogen containing decontamination solutions, in the form of the Decontamination-Anti-corrosion (DANC) compound. This organic solution was designed to provide chemical or biological agent decontamination. In the DANC, a hydantoin ring provides for control of the solubility and inhibition of the corrosive behavior of halogens while still leaving the halogens in a bio-available state. Later it was disclosed that such organic compounds could be attached to organic polymers. Examples of this include the attachment of hydantoin rings to polystyrene, which is disclosed in U.S. Pat. No. 5,490,983, involving polymeric (organic) carriers, such as polystyrene and the heterocyclic structure (hydantoin).

Due to the energetic behavior of reactive molecules and elements, such as halogens, undesired reactions can take place within polymers, which thus degrade the polymer matrix. Further, it has been suggested that the close proximity of halogens to a polymer will result in an increase in the photo-degradability of the associated polymer films.

### SUMMARY OF THE DISCLOSURE

Embodiments of this disclosure overcome prior instability problems of decontaminating compositions that have reactive moieties attached to polymer matrices. In particular, embodiments disclosed herein relate to compositions in which a decontaminating reactive moiety is indirectly attached to a polymer matrix by first attaching the reactive moiety onto an inorganic platform, and then attaching the functionalized inorganic platform onto the polymer matrix.

Thus, one embodiment encompasses a decontaminating composition -comprising an inorganic nanoscale domain, which comprises an inorganic nanoparticle, and an organic reactive molecule grafted via a linker group onto the inorganic nanoparticle, wherein the inorganic nanoscale domain is attached to and uniformly dispersed within a polymer matrix. The inorganic nanoparticles have attachment sites for binding organic reactive molecules, tethering ligands, oleophillic compounds and other compounds/groups capable of binding to inorganic nanoparticles. In one embodiment, the inorganic nanoparticle is an inorganic ceramic particle, which may be selected from the group consisting of alumina, metal oxide and rare earth metal oxide; and the organic reactive molecule is a heterocyclic ring having at least one nitrogen atom, and comprises a 4- to 7-membered ring, wherein at least 3 members of the ring are carbon, from 1 to 3 members of the ring are nitrogen heteroatoms, from 0 to 1 member of the ring is an oxygen or sulfur heteroatom and from 0 to 2 carbon members comprise a carbonyl group, and wherein the linker is attached to a non-carbonyl carbon member. The heterocyclic ring is activated by reaction with a halogen molecule to form an N-halamine, wherein at least one nitrogen heteroatom is joined to a chlorine or bromine moiety.

Embodiments herein also provide methods to phase transition inorganic nanoscale domains (characteristically hydrophilic,) such that it is compatible with an oleophillic polymer matrix.

Embodiments herein also provide methods for decontaminating chemical and/or biological agents comprising contacting an environment containing the hazardous chemical or biological agent with a decontaminating composition comprising an inorganic nanoscale domain, which comprises an inorganic nanoparticle, and an organic reactive molecule grafted via a linker group onto the inorganic nanoparticle, wherein the inorganic nanoscale domain is attached to and uniformly dispersed within an polymer matrix. The methods include decontaminating chemical and/or biological agents, such as mustard agents, nerve agents, acetyl-cholinesterase inhibitors, tear gases, psychotomimetic agents, toxins, biofilms, bacteria, fungi, molds, protozoa, viruses and algae.

Further embodiments encompass methods for preparing decontaminating compositions, wherein inorganic nanoscale domains are chemically reacted with long chain oleophillic acids, which renders the hydrophilic nanoparticle oleophillic.

### BRIEF DESCRIPTION OF THE DRAWINGS

FIG. 1 is a representation of an inorganic nanoscale domain having organic reactive molecules and tethering ligands attached to the surface of an inorganic nanoparticle.

FIG. 2 is a representation of one use of the decontaminating composition as a coating in a drinking water pipeline.

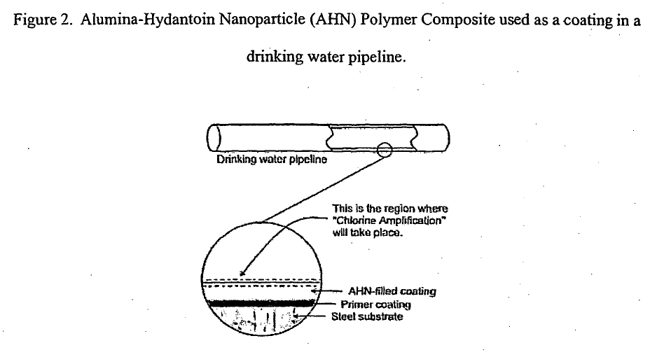

### DETAILED DESCRIPTION OF THE PREFERRED EMBODIMENTS

Embodiments disclosed herein provide novel compositions comprising an inorganic and organic compound, which provides a means for the indirect attachment of a reactive species, such as an organic reactive molecule, within a binder polymer matrix. Such compositions provide a hydrophilic inorganic nanoscale domain that is uniformly dispersed within the polymer matrix. The inorganic nanoscale domain comprises inorganic particles having a nanoscale dimension. Such compositions can enhance the performance potential of the reactive species within the polymer material. The polymer composite that results from the introduction of such reactive species into a polymer matrix provides a self-decontaminating feature. The reactive species include those that are capable of associating with a halogen to form a complex that is active in decontamination of chemical or biological agents. Such capability can be used, for example, as a chlorine amplification medium to resist the formation of biofilms in drinking water systems, as well as for decontaminating/neutralizing other chemical or biological agents.

One embodiment encompasses a highly stable decontaminating composition comprising a inorganic nanoscale domain. The composition comprises an inorganic nanoparticle, and an organic reactive molecule grafted onto the inorganic nanoparticle, wherein the inorganic nanoscale domain is attached to and uniformly dispersed within a polymer matrix. The decontaminating composition can be activated by reaction with a halogen and the resulting halogenated complex is active in chemical or biological decontanination. Accordingly, such embodiment provides encompasses a novel decontaminating composition wherein a reactive organic molecule directly is attached to an inorganic nanoparticle, and in similar fashion, a tethering ligand attaches the entire inorganic nanoscale domain to the binder polymer matrix. In a preferred embodiment, the inorganic nanoscale domain is a nanoscale ceramic domain and the inorganic nanoparticle is an inorganic ceramic nanoparticle.

The inorganic nanoparticle is a platform or surface upon which the organic reactive molecule is attached. The proximity of the reactive species to the inorganic nanoparticle is in the range of 10-100 Angstroms. The inorganic nanoparticle is preferably an inorganic ceramic nanoparticle and is selected from the group of materials that are generally classified as ceramics with preferred materials being alumina, metal oxide and rare earth metal oxide.

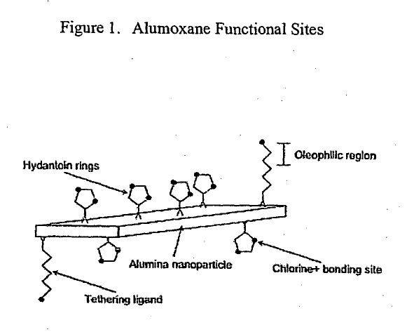

In one embodiment, the nanoscale ceramic domain is a carboxylate-alumoxane. The advantage of the nanoscale size of carboxylate-alumoxane is that it is estimated to have an average of 200 bonding sites per nanoparticle. Accordingly, each nanoparticle will contain a mixture of organic reactive molecules (for decontamination) and tethering ligands (for attaching nanoscale ceramic domain to polymer matrix). FIG. 1 illustrates an alumina nanoparticle having hydantoin organic reactive molecules and tethering ligands attached. In addition, each nanoparticle has a size in the range of 50×50 nm by 1 nm thick, which is considerably smaller than the particle size of conventional pigments, which range typically from 240 microns. The smaller particle size of the nanoparticles provide for a higher surface area and thus, the potential of higher loading of reactive moieties.

Carboxylate-alumoxanes, also known as carboxylato-alumoxanes, are inorganic-organic hybrid materials that contain a boehmite-like ([AlO(OH)]ₙ) aluminum oxygen core, to whose surfaces are attached covalently bound carboxylate groups (i.e., RCO₂⁻, where R=alkyl or aryl group) (Landry et al., J. Mater. Chem., 3:597 (1995)). The carboxylate groups are attached to the aluminum-oxygen surface through bidentate bonding of the carboxylate group to two aluminum atoms on the surface of the boebmite particle. The properties and processability of the carboxylate-alumoxanes are strongly dependent on the nature and size of the attached organic groups. Until recently, carboxylate-alumoxanes were not very useful as processable precursors because they were difficult to prepare. Prior to discovery of a new synthetic route (Apblett et al., Reprinted from Chemistry of Materials, 4 (1992)), carboxylate-alumoxanes were prepared by the reaction of pyrophoric organo-aluminum (e.g., triethylaluminum) with carboxylic acids (Kimura, Y. et al., Coordination Structure of the Aluminum—Atoms of Poly(Methylaloxane), Poly(Isopropoxylaloxane) and Poly[(Acyloxy)Aloxane]; 9 Polyhedron 23:371-76 (1990)) and (Pasynkiewicz, S.; Alumoxanes: Synthesis, Structures, Complexes and Reactions; 9 Polyhedron 23:429-53 (1990)). The high cost of the organometallic compounds and the difficulty of handling highly reactive materials provided a high barrier to the use of carboxylate-alumoxanes as materials for improving the properties of thermoset polymers.

The carboxylate-alumoxanes disclosed herein may be prepared by the reaction of boehmite or pseudoboehmite with an organic reactive molecule containing a carboxylic acid group in a suitable solvent. In addition to the carboxylate groups, the organic reactive molecule containing a carboxylic acid group also may contain terminal a heterocyclic ring.

The boehmite (or pseudoboelunite) source can be a commercial boehmite product such as Catapal (A, B, C, D, or FI, Condea-Vista Chemical Company), boehmite prepared by the precipitation of aluminum nitrate with ammonium hydroxide and then hydrothermally treated at 200° C. for 24 hours, or boehmite prepared by the hydrolysis of aluminum trialkoxides followed by hydrothermal treatment at 200° C. Preferred methods for the preparation of the pseudoboehmite or boehmite particles are those that produce particle sizes of the carboxylate-alumoxanes below 1000 nm and more preferably below 100 nm, and most preferably below 60 nm.

The reaction of the pseudoboehmite (or boehmite) with the organic reactive molecule containing a carboxylic acid group can be carried out in either water or a variety of organic solvents (including, but not limited to alcohols and diols, such as ethylene glycol). However, it is preferable to use water as the solvent so as to the minimize the production of environmentally problematic waste. In a typical reaction, the organic reactive molecule containing a carboxylic acid group is added to boehmite or pseudoboehmite particles, the mixture is heated to reflux, and then stirred for a period of time. The water is removed and the resulting solids are collected. The solids can be re-dispersed in water or other solvents in which the alumoxane and other polymer precursor components are soluble, provided that such redispersion restores the nanoscale medium. It may not necessary to remove the water if the functionalized alumoxanes are to be used in waterborne resin-based polymerization reactions.

The solubility of the carboxylate alumoxanes is dependent only on the identity of the carboxylic acid residue, which includes the organic reactive molecules of the present disclosure, providing it contains a reactive substituent that reacts with the desired co-reactants. The solubilities of the carboxylate-alumoxanes are therefore readily controllable, so as to make them compatible with any desired co-reactants.

In another embodiment, the nanoscale ceramic domain is an alkyl-alumoxane. Alkylalumoxanes are oligomeric aluminum compounds, which can be represented by the general formulae [(R)Al(O)]ₙ and R[(R)Al(O)]ₙ AlR₂. In these formulae, n is an integer and R is straight or branched (C₁-C₁₂)-alkyl, preferably selected from the group consisting of methyl, ethyl, n-propyl, isopropyl, n-butyl, isobutyl, tert-butyl, or pentyl, and having an organic reactive moiety attached. Such compounds can be derived from the hydrolysis of alkylaluminum compounds, AlR₃. It should be noted that while “alkylalumoxane” is generally accepted, alternative terms are found in the literature, such as: alkylaluminoxane, poly(alkylalumoxane), poly(alkylaluminum oxide), and poly(hydrocarbylaluminum oxide). As used herein, the term alkylalumoxane is intended to include all of the foregoing.

Alkylalumoxanes have been prepared in a variety of ways. They can be synthesized by contacting water with a solution of trialkylaluminum, AlR₃, in a suitable organic solvent such as an aromatic or an aliphatic hydrocarbon. Alternatively, a trialkylaluminum can be reacted with a hydrated salt such as hydrated aluminum sulfate. In both cases, the reaction is evidenced by the evolution of the appropriate hydrocarbon, i.e., methane (CH₄) during the hydrolysis of trimethylaluminum (AlMe₃). While these two routes are by far the most common, several “non hydrolysis” routes have been developed.

Conceptually, the simplest route to alkylalumoxanes involves the reaction of water with a trialkylaluminum compound. Simply reacting water or ice (Winter et al., Macromol. Symp., 97:119 (1995)) with an aromatic or aliphatic hydrocarbon solution of a trialkylaluminum will yield an alkylalumoxane.

There is also a wide range of non-hydrolysis reactions that allow for the formation of alkylalumoxanes. Ziegler in 1956 first reported the formation of an alumoxane from the reaction of triethylaluminum with CO₂. Similar product is formed from the reaction of aluminum alkyls with carboxylates and amides (Harney et al., Aust. J. Chem., 27:1639 (1974)). Alkylalumoxanes may also be prepared by the reaction of main group oxides (Boleslawski et al., Organomet. Chem., 97:15 (1975)), while alkali metal aluminates formed from the reaction of trialkylaluminum with alkali metal hydroxides react with aluminum chlorides to yield alkylalumoxanes (Ueyama et al., Inorg. Chem., 12:2218 (1973)).

The reactive species is the active component of the composition that facilitates chemical or biological decontamination and is an organic reactive molecule. In one embodiment, the organic reactive molecule contains a heterocyclic ring having at least one nitrogen atom. The heterocyclic ring comprises a 4- to 7-membered ring, preferably a 5- to 6-membered ring, wherein at least 3 members of the ring are carbon, from 1 to 3 members of the ring are nitrogen heteroatoms, from 0 to 1 member of the ring is an oxygen or sulfur heteroatom and from 0 to 2 carbon members comprise a carbonyl group, and wherein the linker is attached to a non-carbonyl carbon member. The reactive species is activated and ready for chemical or biological decontamination when it reacts with a halogen, such as chlorine or bromine, to form a halogenated complex (e.g., a halogen-charged hydantoin). The heterocyclic rings attract halogens and concentrate them in such a way that the halogen remains available for chemical or biological decontamination. Accordingly, in one embodiment, the heterocyclic ring is activated by reaction with a halogen molecule to form an N-halamine, wherein at least one nitrogen heteroatom is joined to a chlorine or bromine moiety. It is also understood by one of ordinary skill in the art that a halogen can react with a heteroatom other than nitrogen, such as S, O or P, in the heterocyclic ring to form an activated complex. Thus, the disclosure also encompasses decontaminating compositions containing a heterocyclic ring that may or may not have a nitrogen heteroatom, upon which reaction with a halogen forms an activated composition having an S-halogen, O-halogen and/or P-halogen association.

The organic reactive molecule does not directly attach to the polymer matrix. One end of the organic reactive molecule attaches to the inorganic nanoparticle, preferably an inorganic ceramic nanoparticle, while the other end of the organic reactive molecule is free to react with a halogen molecule to form a halogen-activated complex.

Preferred organic reactive molecules contain a heterocyclic ring selected from the group consisting of a pyrrolidinone, pyrrolidone dione, triazolidinone, oxazolidinone, oxazolidine dione, thiazolidinone, thiazolidine dione, hydantoin, triazinone, triazine dione, imidazolidinone, imidazolidine dione, pyrimidinone, pyrimidine dione, oxazinone, dihydro-oxazinone, dihydro-oxazine dione, dihydro-thiazinone, dihydro-thiazine dione, thiazinone, oxazinanone, oxazinane dione, thiazinanone, thiazinane dione, oxadiazinanone, oxadiazinane dione, thiadiazinanone, thiadiazinane dione, azepanone, azepane dione, azepane trione, oxazepanone, oxazepane dione, oxazepane trione, thiazepanone, thiazepane dione, thiazepane trione, diazepanone, diazepane dione, diazepane trione, oxadiazepanone, oxadiazepane dione, oxadiazepane trione, thiadiazepanone, thiadiazepane dione, thiadiazepane trione, triazepanone, triazepane dione, triazepane trione, oxatriazepanone, oxatriazepane dione, oxatriazepane trione, thiatriazepanone, thiatriaepane dione, thiatriazepane trione, a dihydro derivative thereof, and a tetrahydro derivative thereof. More preferably, the heterocyclic ring is selected from the group consisting of a hydantoin, triazine dione, imidazolidinone, and pyrimidine.

As mentioned above, the organic reactive molecule contains a linker group that attaches the heterocyclic ring to the inorganic nanoparticle, wherein the linker group is attached to a non-carbonyl carbon member of the heterocyclic ring. The linker group is selected from the group consisting of (C₁-C₁₂)-carboxyl group and (C₁-C₁₂)-alkoxy group, as well as (C₁-C₁₂)-alkyl groups having amide, amine, thiol, and other S or N-based moieties. The linker molecule preferably is a (C₁-C₆)-carboxyl group, more preferably a (C₁-C₃)-carboxyl group. In a preferred embodiment, the organic reactive molecule is selected from the group consisting of

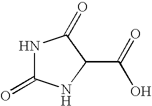

### 2,4-dioxoimidazolidone-5-acetic acid or

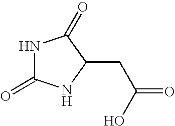

### 2,4-dioxoimidazolidone-5-carboxylic acid.

In embodiments of this disclosure, the inorganic nanoscale domain is attached to the polymer matrix by a tethering ligand. The tethering ligand provides a molecular bridge to link the hydrophilic surface of the inorganic nanoscale domain to the oleophillic surface of the polymer matrix. The length of the tethering ligand can be varied to control the spacing or distance between the inorganic nanoscale domain and the polymer matrix, a design parameter that one of ordinary skill in the art may customize for targeted composite designs. The tethering ligand is selected from the group consisting of an amino acid, (C₁-C₁₂)-alkylamino alcohol, (C₁-C₁₂)-alkylamino ester, (C₁-C₁₂)-alkyl diol, (C₁-C₁₂)-alkyldiamine, (C₁-C₁₂)-alkyl diester, (C₁-C₁₂)-alkyl acid, (C₁-C₁₂)-alkanol ester, (C₁-C₁₂)-alkyl acid ester, (C₁-C₁₂)-alkanol acid, (C₁-C₁₂)-alkyl diamide, (C₁-C₁₂)-alkyl amine amide, (C₁-C₁₂)-alkyl acid amide, (C₁-C₁₂)-alkyl ester amide and (C₁-C₁₂)-alkanol amide. Preferably, the tethering ligand is an amino acid, such as lysine, taurine, arginine, glutamic acid, aspartic acid and asparagine.

Accordingly, in embodiments of this disclosure, the inorganic nanoscale domain is prepared first before attachment to the polymer matrix. Inorganic nanoscale domains, such as nanoscale ceramic domains (e.g., carboxylate-alumoxanes), have high surface area that provide a high number of bonding sites. The number of organic reactive molecules and tethering ligand may be varied, depending on the applicable design considerations, and the ratio of organic reactive molecules to tethering ligand can be from about 200:1 to about 20:1.

In one embodiment, the nanoscale ceramic domains may be prepared by first reacting alumina with a desired amount of the organic reactive molecule and followed by reacting with a desired amount of the tethering ligand. Alternatively, the alumina first may be reacted with the tethering ligand followed by reaction with the organic reactive molecule. The nanoscale ceramic domains may be prepared by in situ reaction of alumina, organic reactive molecule and tethering ligand, i.e., by simultaneous reaction with alumina, organic reactive molecule and tethering ligand.

In another embodiment where it is desirable to achieve uniform dispersion of inorganic nanoparticles within a polymer matrix, the inorganic nanoscale domains can be tethered to the polymer matrix in the presence of an additional oleophillic compound, such as stearic acid or another (C₁₂-C₂₅)-straight chain carboxylic acid, to enhance the oleophillicity of the resulting inorganic nanoscale domains. Thus, in one embodiment, such oleophillic compound binds to bonding sites of the inorganic nanoparticle. Increasing the chain length of the oleophillic compound will increase the overall oleophillicity of the inorganic nanoparticle. The oleophillic compound can be introduced during any step of the preparation of the decontaminating composition. For example, the oleophillic compound may be added before, during and after the step of attaching the tethering ligand and/or the organic reactive molecule to the inorganic nanoparticle. The oleophillic compound may also be added during the step of attaching the inorganic nanoscale domain to the polymer matrix.

The ratio of reactants, such as inorganic nanoparticles, reactive organic molecules, tethering agents and oleophillic compounds can vary depending on the type and nature of each reactant, and will be readily ascertainable by one of ordinary skill in the art without undue experimentation.

The polymer matrix to which the inorganic nanoscale domain is attached may be organic or inorganic. Inorganic polymer matrices include silica and silicate-based polymers, metal oxides (e.g., zinc oxide, indium-tin oxide, ferrite and the like), and ceramic matrices. A suitable organic polymer matrix is selected from the group consisting of epoxides, phenol-formaldehyde (phenolic) resins, polyamides (nylons), polyesters, polyimides, polycarbonates, polyurethanes, quinone-amine polymers, acrylates, polyacrylics and polyolefins. The polymer matrix may be formed separately from the inorganic nanoscale domain. Thus in one embodiment, the polymer matrix is pre-formed/pre-polymerized prior to tethering the inorganic nanoscale domain thereto.

While not wishing to be bound by any particular theory, it is believed that compositions according to the embodiments disclosed herein may exhibit one or more of the following desirable characteristics.

The first is the approach to highly efficient deployment of the reactive species within a polymer material. Through particle size reduction from micron to nanoscale, the inorganic nanoparticles provide an inorganic surface that has an extremely large surface area. There is thus a large inorganic surface area within the organic polymer phase.

A second feature is the behavior of the inorganic surface as a means to mitigate polymer degradation. The reactive species is not directly attached to a polymer phase, so that the active species are distanced from the binder polymer phase. As a result there is less concern for the damaging effect of highly energetic reactive molecules, such as the chlorine cation, on the proximate polymer. The preferred inorganic ceramic nanoparticle composition can be determined by considering the material characteristics, such as Gibbs free energy and accordingly, materials, which are resistant to attack due to the ceramic property can be identified.

A third feature is that the surface of the nanoscale ceramic domain is hydrophilic. The use of an inorganic ceramic nanoparticle, such as alumoxane, has high surface energy. This facilitates the charging and discharge of the reactive species, which is essential to a recharge characteristic of the reactive species.

The fourth feature is the relative proximity of the individual reactive species to each other on the inorganic nanoscale domain. This is made possible by the inorganic nanoscale domains that exist within a polymer matrix. The transport of charging media along the nanoparticle domains results in a diffusion rate that is faster than normal Fickian diffusion and is akin to an ionic enhanced vacancy diffusion mechanism where the reactive species site penetrates the polymer matrix by passing to vacant bonding sites.

This disclosure also encompasses a method for decontaminating chemical or biological agents comprising contacting an environment containing the hazardous chemical or biological agent with the decontaminating composition. Chemical or biological warfare agents can include, inter alia, mustard agents, nerve agents, acetyl-cholinesterase inhibitors, tear gases, psychotomimetic agents, toxins, biofilms, bacteria, fungi, molds, protozoa, viruses and algae. Particular chemical or biological warfare agents include Tabun ((CH₃)₂N—P(═O)(—CN)(—OC₂H₅)), Sarin (CH₃—P(═O)(—F)(—OCH(CH₃)₂)), Soman (CH₃—P(═O)(—F)(—CH(CH₃)C(CH₃)₃), cyclohexyl methylphosphonofluoridate/GF (CH₃—P(═O)(—F)(cyclo-C₆H₁₁)), O-ethyl S-diisopropylaminomethyl methylphosphonothiolate/VX (CH₃—P(═O)(—SCH₂CH₂N[CH(CH₃)₂]₂)(—OC₂H₅)), Vibrio cholera, Staphylococcus, Pseudomonas, Salmonella, Shigella, Legionella, Methylobacterium, Klebsiella, and Bacillus, Candida, Rhodoturula, mildew, Giardia, Entamoeba, Cryptosporidium, poliovirus, rotavirus, HIV virus, herpesvirus, Anabaena, Oscillatoria, Chlorella, and sources of biofouling in closed-cycle cooling water systems. In the case of biological warfare agents, the following can be decontaminated: Bacillus anthracis (anthrax), Clostridium botulinum (botulinum toxins), Brucella melitensis, Brucella abortus, Brucella suis, and Brucella canis (brucellosis), Vibrio cholera (cholera), clostridium perfringens toxins, congo-crimean hemorrhagic fever virus, ebola baemorrhagic fever virus, Pseudomonas pseudomallei (meliodosis), Yersinia pestis (plague), Xenopsylla cheopis (plague), Pulex irritans (plague), Coxiella burnetii (Q fever), ricin, Rift Valley Fever Virus, saxitoxin, smallpox virus, Staphylococcus aureus (Staphylococcal Enterotoxin B), trichothecene mycotoxins, Francisella tularensis (Tularemia), and Venezuelan equine encephalitis).

The functional nanoparticle species can be incorporated into decontaminating compositions intended for use as decontaminating chemical or biological agents in a variety of environments, including aqueous and other solution media, semi-solid media, surfaces of materials and in gas streams by treating the media or material with an effective amount of decontaminating composition. The decontaminating compositions serve to decontaminate chemical or biological agents during and after a chemical or biological event. An aqueous medium can include, for example, that as found in potable water sources, swimming pools, hot tubs, industrial water systems, cooling towers, air conditioning systems, waste disposal units and the like. As used herein, a “liquid or semi-solid medium” includes liquid or semi-solid media in which halogen-sensitive chemicals or microorganisms can reside, which can include, paint, wax, household cleaners, wood preservatives, oils, ointments, douches, enema solutions and the like.

As used herein, a “surface” can include any surface upon which halogen-sensitive chemicals or microorganisms can reside and to which the decontaminating composition can be bound, which can include surfaces of, for example, fabric material (e.g., cellulose or synthetic fiber), filter material, membranes (e.g., porous organic membranes, including poly(ether-ether ketone) (“PEEK”) membranes and PEEK membranes having a urethane modification), metal, rubber, concrete, wood, glass, coating and bandaging. In one embodiment, the decontaminating composition is bound to a pipe or tank surface for the control of microorganisms, such as Vibrio Cholera and other pathogenic bacteria, that live in biofilm (durable slime layer) in municipal water systems. FIG. 1 depicts one mechanism for utilizing this technology to prevent biofilm formation at pipe and tank surfaces. Chlorine disinfection by-products are carcinogenic and it is desirable to reduce chlorination level. The decontaminating composition is capable of amplifying halogen (e.g., Cl or Br) concentration in the surface region of the polymer matrix, which utility as a biofilms-mitigating agent can be optimized versus the chlorine concentration generally found in municipal water supplies. Thus, the chlorine concentration in municipal water supplies are reduced in the presence of the decontaminating composition. The chlorine amplification feature results in a surface where bacteria cannot attach and survive. In addition, chlorine in the municipal water supplies provides a continuous recharge of deactivated decontaminating composition.

As used herein, “a gaseous medium” includes any gas in which halogen-sensitive chemicals or microorganisms can reside, such as air, oxygen, nitrogen, or any other gas, such as found in air handling systems in, for example, enclosed bunkers, vehicles, hospitals, hotels, convention centers or other public buildings.

For aqueous, liquid or gas media, decontamination is best done by flowing chemically or biologically contaminated water or gas, e.g., air, over or through the solid polymer in an enclosed column or cartridge or other type filter. The residence time of the contaminated substance in the filter unit will determine the efficacy of decontamination. For decontamination applications involving paints, coatings, preservatives and semi-solid media, the decontaminating compositions are best introduced as fine suspensions in the base materials to be decontaminated. These decontaminating compositions can be incorporated into textile fibers, rubber materials, and solid surfaces, as well to serve as chemical or biological preservatives.

Once a decontaminating composition becomes ineffective in neutralizing chemical or biological agents due to inactivation of the N—Cl or N—Br moieties, it can be regenerated by passing an aqueous solution of free halogen through it. Additionally, the decontaminating composition can be created or regenerated in situ by adding a stoichiometric amount of free halogen, either chlorine or bromine, to a precursor reaction mixture to form the decontaminating composition contained in the desired material, such as in a filter unit, in paint, oil, textile fabric or the like, or bound to a surface of a material such as wood, glass, plastic polymer coating, textile fabric, metal, rubber, concrete, cloth bandage or the like.

Thus, the unhalogenated decontaminating composition can be incorporated into a material, surface, or filter unit as described above, and can then later, at an advantageous time, be halogenated in situ to render it active for chemical or biological decontamination. In one embodiment, such a material, surface, or filter unit can be a replaceable item that can be reactivated after replacement with a fresh unit. In other embodiments, the item may be disposable.

The decontaminating compositions described herein can also be employed together with sources of active disinfecting halogen, such as free chlorine or bromine or the various N-halamine sources of the same. The decontaminating compositions liberate very little free halogen themselves and they can be used to abstract larger amounts of free halogen from water flowing through them. They can serve as a source of small amounts of free halogen residual for decontamination applications.

The decontaminating compositions described herein can be employed in a variety of chemical or biological decontamination applications. They will be of importance in controlling chemical or biological contamination in cartridge or other type filters installed in the recirculating water systems of remote potable water treatment units, swimming pools, hot tubs, air conditioners, and cooling towers, as well as in recirculating air-handling systems used in military bunkers and vehicles and in civilian structures. For example, the decontaminating compositions will prevent the growth of undesirable microorganisms, such as the bacteria genera Staphylococcus, Pseudomonas, Salmonella, Shigella, Legionella, Methylobacterium, Klebsiella, and Bacillus; the fungi genera Candida, Rhodoturula, and molds such as mildew; the protozoa genera Giardia, Entamoeba, and Cryptosporidium; the viruses poliovirus, rotavirus, HIV virus, and herpesvirus; and the algae genera Anabaena, Oscillatoria, and Chlorella; and sources of biofouling in closed-cycle cooling water systems. They will be of particular importance to the medical field for use in ointments, bandages, feminine napkins and tampons, sterile surfaces, condoms, surgical gloves, and the like, and for attachment to liners of containers used in the food processing industry. They can be used in conjunction with textiles for sterile applications, such as coatings on sheets or bandages used for burn victims or on microbiological decontamination suits.

The decontaminating compositions may have direct application to the military, firefighting and emergency response personnel who must face chemical and/or biological hazards. Such applications can include use of such compositions in or on clothing (including gloves, masks, boots and other footwear, undergarments), gear, respirators or breathing devices etc.

The decontaminating compositions described herein can be used in diverse liquid and solid formulations such as powders, granular materials, solutions, concentrates, emulsions, slurries, and in the presence of diluents, extenders, fillers, conditioners, aqueous solvent, organic solvents, and the like.

### EXAMPLES

The following examples are presented to illustrate the ease and versatility of the approach and are not to be construed as in any way limiting the scope of this disclosure.

### Example 1

### Composite sample incorporating biologically reactive organic molecule affixed alumina nanoparticle.

| NANOSOL SOLUTION | QUANTITY IN GRAMS |
|---|---|
| Deionized water | 100 |
| Dispal Alumina | 11 |
| Lactic Acid | 1 |

| FUNCTIONALIZATION SOLUTION | QUANTITY IN GRAMS |
|---|---|
| Hydantoin-5-Acetic Acid (95% in | 5 g (in 20 g water) |
| deionized water) | |
| Lysine (25% solution in deionized water) | 11 |

The following exemplary composition is provided. The nano-sol solution ingredients were placed under shear agitation using a high-speed dissolver blade. Temperature of the sol was held at 126F±5 F. Vacuum was maintained at 15″ Hg. The functionalization solution was introduced drop-wise into the mixture over a period of approximately one hour.

The chemical reaction was evidenced by an exothermic reaction, where the temperature rose to a peak of 135 F, at which, point the vacuum was discontinued and the mixture was allowed to cool to approximately 115 F under slow agitation.

The heating was restored and temperature raised to 135 F with vacuum level set at 12″ Hg. These conditions resulted in removal of excess water from the solution. When approximately 30% of the water (by weight) had been removed, the process was terminated, and the solution was filtered and packaged. The resulting nano-sol solution contained the novel bio-reactive compound in water. The pH of the resulting solution was approximately 5.

Next, 11 grams of the functionalized nano-sol solution was mixed with 10 grams of epoxy emulsion (AP-550 manufactured by Air Products and Chemicals, Inc.) to form a coating containing the novel compound. The coating was spread evenly over a release film using a 10-mil drawdown bar; and allowed to cure for 48 hours under ambient conditions. The mixture cured to form a tough and flexible coating film.

After curing for 48 hours, the resultant film was next tested for kinetic chlorine transport. The kinetic behavior testing protocol provided preliminary insight into charging efficacy and chlorine binding kinetics within the coating.

The “free film” candidate coating sample provided a barrier between the charging solution and the indicator solution. The charge rate is quantified by tracking the time required for diffusion of the chlorine through the coating specimen. It has been shown that baseline (reference) coatings (having no hydantoin component) have high resistance to chlorine diffusion. It is postulated that the chlorine transfer mechanism through the hydantoin-loaded coating is, in fact, not a traditional Fickian diffusion process, but rather it is controlled by a mechanism whereby the chlorine atoms move from one hydantion to the next. The testing has shown that “diffusion” of chlorine through a hydantoin-loaded, 175 micron (thickness) coating will occur within ½ hour. The same coating without hydantion takes days for the chlorine to traverse. The testing has also demonstrated a direct correlation between the rate of chlorine diffusion and the hydantoin loading in the coating, and further permits evaluation of performance over a wide range of parameters. The result is a very effective visual indication of this “end point” which can easily be correlated with time. By means of this very simple apparatus, useful kinetics data can be generated.

Observations:

| TIME | OBSERVATION |
|---|---|
| 0 mins. | Start test by filling one chamber with 10% KI (yellow) |
| | indicator solution and opposite chamber with 20% Clorox |
| | solution (both diluted with deionized water). |
| 10 mins. | Yellow color indication along the air/water interface of the |
| | test film. |
| 45 mins. | Progression of color change on film surface. |
| 105 mins. | Uniform color change across coating/indicator contact surface, |
| | extending to approximately 100 microns above the liquid/air |
| | interface. |

For comparison purposes, a film was prepared using the above-described protocol, with the sole exception that no hydantoin-5-acetic acid was introduced. When tested using the diffusion cell, there was no color change over 48 hours.

### Example 2

Composite sample incorporating stearic acid to provide phase transition feature to the alumina nanoparticle.

| NANOSOL SOLUTION | QUANTITY IN GRAMS |
|---|---|
| Deionized water | 275 grams |
| Dispal Alumina | 60 grams |
| Lactic acid | 10 grams |

| FUNCTIONALIZATION SOLUTION | QUANTITY IN GRAMS |
|---|---|
| 50% Lysine solution | 36 grams |
| deionized water | 150 grams |
| steatic acid | 25 grams |

The following exemplary composition is provided. Upon initiation of the process, alumina, lactic acid and the stearic acid are combined to form a viscous solution. After standing overnight, the solution loses much of this viscosity and becomes an easily pumpable solution.

The nano-sol solution ingredients were placed under shear agitation using a high speed dissolver blade. Temperature of the sol was initially maintained in the range of 175 F. The functionalization solution was transferred into the nano-sol solution at a dropwise addition rate of approximately 60 mL per hour. A peristaltic pump provides a controlled transfer.

After addition of approximately 100 mL of the functionalization solution, the viscosity of the mixture became excessively high. Addition of 50 grams of water and 10 grams of additional lactic acid brought the viscosity of the mixture to an acceptably fluid state. At this point the pH is in range of 3-4.

Temperature was reduced to 150 F and the transfer of functionalization solution was reinstated. After addition of approximately 300 mL of functionalization solution, an additional 100 mL of water was added.

When the addition of functionalization solution was completed, the shear rate was increased by raising the dissolver blade's speed range to maximum, and a vacuum was applied. The temperature of the mixture was maintained at approximately 155 F. These conditions were maintained for approximately two and one half hours.

The process was then discontinued to permit weighing of the net contents of materials in the mixing vessel. Net weight of the materials was determined to be 472 grams.

300 grams of binder polymer (Jeffamine D-2000, as manufactured by Huntsman Chemical) was added to the reaction mixture. It was observed that the resultant mixture was compatible and resulted in a readily flowable mixture. The processing continued under vacuum and elevated temperature, until all of the water was removed. Some increase in viscosity was observed as the removal of the water approached the endpoint, whereupon only the organic phase remained and the viscosity became remarkably reduced.

The resin solution was incorporated into a standard polyurea coating formulation and drawn down using standard techniques to yield a 20 mm thick coating film that could be used for testing purposes. The result of the alumoxane nanoparticle introduction in this manner is a coating that has improved resistance to oxygen permeation.

1. A decontaminating composition comprising an inorganic nanoscale domain, which comprises an inorganic nanoparticle, and an organic reactive molecule grafted onto the inorganic nanoparticle.

2. The composition of claim 1 wherein the decontaminating composition is uniformly dispersed within a polymer matrix.

3. The composition of claim 1, wherein the inorganic nanoparticle is an inorganic ceramic nanoparticle selected from the group consisting of alumina, metal oxide and rare earth metal oxide.

4. The composition of claim 1, wherein the inorganic nanoscale domain is a carboxylate-alumoxane or an alkyl-alumoxane.

5. The composition of claim 1, wherein the organic reactive molecule is not attached to the polymer matrix.

6. The composition of claim 1, wherein the organic reactive molecule is a heterocyclic ring having at least one nitrogen atom.

7. The composition of claim 1, wherein the heterocyclic ring comprises a 4- to 7-membered ring, wherein at least 3 members of the ring are carbon, from 1 to 3 members of the ring are nitrogen heteroatoms, from 0 to 1 member of the ring is an oxygen or sulfur heteroatom and from 0 to 2 carbon members comprise a carbonyl group, and wherein the linker is attached to a non-carbonyl carbon member.

8. The composition of claim 6, wherein the heterocyclic ring is selected from the group consisting of a pyrrolidinone, pyrrolidone dione, triazolidinone, oxazolidinone, oxazolidine dione, thiazolidinone, thiazolidine dione, hydantoin, triazinone, triazine dione, imidazolidinone, imidazolidine dione, pyrimidinone, pyrimidine dione, oxazinone, dihydro-oxazinone, dihydro-oxazine dione, dihydro-thiazinone, dihydro-thiazine dione, thiazinone, oxazinanone, oxazinane dione, thiazinanone, thiazinane dione, oxadiazinanone, oxadiazinane dione, thiadiazinanone, thiadiazinane dione, azepanone, azepane dione, azepane trione, oxazepanone, oxazepane dione, oxazepane trione, thiazepanone, thiazepane dione, thiazepane trione, diazepanone, diazepane dione, diazepane trione, oxadiazepanone, oxadiazepane dione, oxadiazepane trione, thiadiazepanone, thiadiazepane dione, thiadiazepane trione, triazepanone, triazepane dione, triazepane trione, oxatriazepanone, oxatriazepane dione, oxatriazepane trione, thiatriazepanone, thiatriaepane dione, thiatriazepane trione, a dihydro derivative thereof, and a tetrahydro derivative thereof.

9. The composition of claim 7, wherein the heterocyclic ring is selected from the group consisting of a hydantoin, triazine dione, imidazolidinone, and pyrimidine.

10. The composition of claim 8, wherein the hydantoin is a 2-4-dioxoimidazolidone and the linker group is attached in the 5-position of the hydantoin.

11. The composition of claim 9, wherein the heterocyclic ring is activated by reaction with a halogen molecule to form an N-halamine, wherein at least one nitrogen heteroatom is joined to a chlorine or bromine moiety.

12. The composition of claim 1, wherein the organic reactive molecule contains a (C₁-C₁₂)-carboxyl linker group or (C₁-C₁₂)-alkoxy linker group that attaches the organic reactive molecule to the inorganic ceramic nanoparticle.

13. The composition of claim 11, wherein the linker group is a (C₁-C₆)-carboxyl group.

14. The composition of claim 1, wherein the organic reactive molecule contains a carboxylic acid linker group.

15. The composition of claim 14, wherein the organic reactive molecule is selected from the group consisting of an amino acid, (C₁-C₁₂)-alkylamino alcohol, (C₁-C₁₂)-alkylamino ester, (C₁-C₁₂)-alkyl diol, (C₁-C₁₂)-alkyldiamine, (C₁-C₁₂)-alkyl diester, (C₁-C₁₂)-alkyldiacid, (C₁-C₁₂)-alkanol ester, (C₁-C₁₂)-alkyl acid ester, (C₁-C₁₂)-alkanol acid, (C₁-C₁₂)-alkyl diamide, (C₁-C₁₂)-alkyl amine amide, (C₁-C₁₂)-alkyl acid amide, (C₁-C₁₂)-alkyl ester amide and (C₁-C₁₂)-alkanol amide.

16. The composition of claim 15, wherein the amino acid is lysine and taurine.

17. A method for decontaminating chemical or biological agents comprising contacting an environment containing the chemical or biological agent with a decontaminating composition according to claim 1.

18. The method of claim 17, wherein the decontaminating composition reacts with and decontaminates a chemical or biological warfare agent.

19. The method of claim 18, wherein the chemical or biological warfare agent is selected from the group consisting of mustard agents, nerve agents, acetyl-cholinesterase inhibitors, tear gases, psychotomimetic agents, toxins, biofilms, bacteria, fungi, molds, protozoa, viruses and algae.

20. The method of claim 19, wherein the chemical or biological warfare agent is selected from the group consisting of Bacillus anthracis, Clostridium botulinum, Brucella melitensis, Brucella abortus, Brucella suis, and Brucella canis, Vibrio cholera, clostridium perfringens toxins, congo-crimean hemorrhagic fever virus, ebola haemorrhagic fever virus, Pseudomonas pseudomallei, Yersinia pestis, Xenopsylla cheopis, Pulex irritans, Coxiella burnetii, ricin, Rift Valley Fever Virus, saxitoxin, smallpox virus, Staphylococcus aureus, trichothecene mycotoxins, Francisella tularensis, and Venezuelan equine encephalitis.

21-33. (canceled)
